(19) United States
(12) Patent Application Publication
(10) Pub. No.: US 2011/0218172 A1
(43) Pub. Date: Sep. 8, 2011

(54) COMPOSITION FOR DOWN-REGULATING PRO-INFLAMMATORY MARKERS

(76) Inventors: Muhammed Majeed, East Windsor, NJ (US); Anjali Pandey, Bangalore (IN); Sarang Bani, Babgalore (IN); Beena Bhat, Bangalore (IN)

(21) Appl. No.: 12/768,871

(22) Filed: Apr. 28, 2010

Related U.S. Application Data
(60) Provisional application No. 61/309,481, filed on Mar. 2, 2010.

(51) Int. Cl. A61K 31/715; A61P 29/00

(52) U.S. Cl. 514/54

(57) ABSTRACT

The present invention provides a composition for down-regulating pro-inflammatory markers. The composition comprises boswellic acid fraction and polysaccharide faction obtained from Boswellia species at specific concentrations showing enhancement in their activity as compared to boswellic acid fraction and the polysaccharide fraction alone. The invention further comprises use of polysaccharide fraction individually or in combination with boswellic acid fraction for inhibition of PGE2.

This application is a non-provisional application of provisional application 61/309,481 filed on 2 Mar. 2010.

### FIELD OF THE INVENTION

The present invention relates to a composition comprising boswellic acid fraction and polysaccharide fraction obtained from Boswellia species. The composition is synergistic in showing enhanced bioactivity, particularly down-regulating the pro-inflammatory markers.

The invention also relates to use of polysaccharide fraction alone or in combination with boswellic acid fraction for inhibition of PGE2.

### BACKGROUND AND PRIOR ART

Survival is impossible without precise regulation of immune system. The production of pro-inflammatory cytokines is a critical physiological process to orchestrate immune and metabolic responses during development, tissue regeneration, healing, trauma or infection and to protect our bodies against hemorrhage, ischemia, cancer and sepsis. A controlled production of pro-inflammatory cytokines, such as interleukins (ILs), tumour necrosis factor-alpha (TNF-α) triggers beneficial inflammatory responses that promote local coagulation to confine infection and tissue damage (Ulloa and Tracey, 2005). However, the unrestricted production of these cytokines is more dangerous than the original injury and it is one of the principal causes of human morbidity and mortality. One of the most dramatic examples of this process is ‘severe sepsis’, the leading cause of death in intensive care units and one of the principal causes of death in developed societies (Martin et al., 2003). Severe sepsis is characterized by an overwhelming production of proinflammatory cytokines that causes systemic inflammation, cardiovascular dysfunction and lethal multiple organ failure (Van der Poll and Lowry, 1995; Hotchkiss and Karl, 2003; Rice and Bernard, 2005). This effect is illustrated by the studies indicating that neutralizing pro-inflammatory cytokines (monoclonal anti-TNF antibodies and IL-1 receptor antagonists) have proven to be successful in inflammatory conditions such as rheumatoid arthritis, Crohn's disease, ankylosing spondylitis and psoriasis (Feldmann, 2002; Ulloa and Tracey, 2005; Rutgeerts et al., 2006; Ulloa and Messmer, 2006)

In addition to cytokines, other mediators like histamine, prostaglandins, leukotrienes, bradykinin etc also play a role in inflammation. Thus, these serve as markers and are useful in diagnosing disease conditions, especially in those conditions where they are present at elevated levels. It is therefore essential to regulate the markers for precise control of immune system.

The gum resin of Boswellia serrata (N.O. Burseraceae) known as “Dhup”, Indian Frankincense or Indian olibanum has a long history of use in religious ceremonies and in perfumery applications. The health applications of Indian frankincense, long known in the Ayurvedic tradition, have come into focus in the western world, over the last thirty years, resulting in expanded applications of standardized extracts of the gum resin exudates. Such extracts are used as ingredients in dietary supplements and cosmetics to support healthy aging. The most popular application in dietary supplements is in joint health support products to support normal joint functions and mobility.

Recent scientific evidence increasingly supports the healthful effects of Boswellia serrata. Typically, the gum oleoresin exudates of Boswellia serrata is reported to contain sesquiterpenoid essential oils (8-12% w/w), polysaccharides (45-60% w/w), and higher terpenoids (25-35% w/w). The biomarker constituents in the extract of the gum resin are a group of pentacyclic triterpene compounds known as boswellic acids.

Boswellic acids have been shown to inhibit the enzyme 5-lipoxygenase, the enzyme that catalyzes the formation of pro-inflammatory leukotrienes from arachidonic acid. In addition to this mechanism, boswellic acids also decrease the activity of the enzyme, Human Leukocyte Elastase (HLE). This dual action is unique to boswellic acids (Safayhi, H et al., 1997). As leukotriene formation and HLE release are increased simultaneously by neutrophil stimulation in a number of inflammation and hypersensitivity-based human diseases, it is generally believed that the reported blockade of two pro-inflammatory enzymes by boswellic acids, and their beneficial effects on complement proteins and mast cell stabilizing activity could be the rationale for the healthful effects of Boswellia extracts, documented in multiple preclinical and clinical studies. Extracts of Boswellia serrata gum resin are generally resinous in nature, and the biomarker boswellic acids (lipophilic compounds) are insoluble in water.

US2003/0186932 describes a water soluble bioactive fraction isolated from gum resin exudate of Boswellia serrata. It further discloses that a combination of the fraction and boswellic acids in equal ratios (1:1) showed additive effect and not a synergistic effect for anti-arthritic activity.

U.S. Pat. No. 7,582,314 describes a method of treating psoriasis affected individual by administering a composition comprising boswellic acids and elemental selenium.

US20080275117 describes a method of treating arthritis by the use of composition comprising boswellic acids and/or their acetates in an amount greater than 65%. The composition is also said to further comprise polysaccharides and n-octyl acetate, incensole, incensole acetate, linalool, borneol, camphene, elemene, caryophyllene, incensole oxide, incensole oxide acetate or combination thereof.

The present invention encompasses a composition comprising boswellic acid fraction and polysaccharide fraction obtained from Boswellia species showing enhancement in down-regulating/inhibiting the pro-inflammatory markers.

### SUMMARY OF THE INVENTION

The present invention relates to a composition comprising boswellic acid fraction and polysaccharide fraction obtained from Boswellia species for down-regulating pro-inflammatory markers such as TNF-α, IL-1β, Nitric oxide, IFN-γ and LTB4. Boswellic acid fraction is present at a concentration of about 60% and polysaccharide fraction at a concentration of about 40%.

The composition comprising boswellic acid fraction and the polysaccharide fraction shows enhancement in its activity as compared to fractions individually.

The invention further relates to use of polysaccharide fraction alone or in combination with boswellic acid fraction for inhibition of PGE2.

### BRIEF DESCRIPTION OF ACCOMPANYING FIGURES

FIG. 1: Effect of multiple dose of boswellic acid fraction obtained from Boswellia species on extracellular in vivo TNF-α and IL-1β estimation in serum from treated balb/c mice.

FIG. 2: Effect of multiple dose of polysaccharide fraction obtained from Boswellia species on extracellular in vivo TNF-α and IL-1β estimation in serum from treated balb/c mice.

FIG. 3: Effect of multiple dose of the composition on extracellular in vivo TNF-α and IL-1β estimation in serum from treated balb/c mice.

FIG. 4: Effect of multiple dose of boswellic acid fraction obtained from Boswellia species on extracellular in vivo NO estimation in serum from treated balb/c mic.e

FIG. 5: Effect of multiple dose of polysaccharide fraction obtained from Boswellia species on extracellular in vivo NO estimation in serum from treated balb/c mice.

FIG. 6: Effect of multiple dose of the composition on extracellular in vivo NO estimation in serum from treated balb/c mice.

FIG. 7: Comparative anti-arthritic activity (prophylactic) of boswellic acid fraction, polysaccharide fraction and the composition in Mycobacterium tuberculli induced inflammatory arthritis in rats (injected paw).

FIG. 8: Expression of TNF-alpha in supernatant from arthritic paw tissue homogenate in Mycobacterium tuberculli induced inflammatory arthritis in rats treated with boswellic acid fraction, polysaccharide fraction and the composition at graded dose levels.

FIG. 9: Expression of PGE2 in supernatant from arthritic paw tissue homogenate in Mycobacterium tuberculli induced inflammatory arthritis in rats treated with boswellic acid fraction, polysaccharide fraction and the composition at graded dose levels.

FIG. 10: Expression of LTB4 in supernatant from arthritic paw tissue homogenate in Mycobacterium tuberculli induced inflammatory arthritis in rats treated with boswellic acid fraction, polysaccharide fraction and the composition at graded dose levels.

FIG. 11: Effect of boswellic acid fraction, polysaccharide fraction and the composition on intracellular IFN-γ expression by flow cytometry in splenocytes of Mycobacterium tuberculli induced inflammatory arthritic animals.

FIG. 12: Anti-arthritic activity (therapeutic) of boswellic acid fraction, polysaccharide fraction and the composition (effective dose) in Mycobacterium tuberculli induced established inflammatory arthritis in rats (injected paw).

FIG. 13: shows the comparison of water solubility of boswellic acid fraction and the composition of present invention.

FIG. 14: flowchart showing the steps of preparation of composition of present invention.

### DETAILED DESCRIPTION OF THE INVENTION

The present invention relates to a synergistic composition comprising boswellic acid fraction at a concentration of about 60% and polysaccharide fraction at a concentration of about 40%. optionally along with pharmaceutically acceptable excipients.

In another embodiment of the present invention, the boswellic acid fraction and the polysaccharide fraction are obtained from Boswellia species.

In yet another embodiment of the present invention, the boswellic acid fraction comprise β-boswellic acid, acetyl-β-boswellic acid, 11-keto-β-boswellic acid and acetyl-11-keto-β-boswellic acid.

In still another embodiment of the present invention, the polysaccharide fraction comprise galactose, arabinose, D-glucuronic acid and 4-O-methyl-glucuronoarabino-galactan.

In still another embodiment of the present invention, the pharmaceutically acceptable excipients are selected from a group comprising antiadherents, binding agents, coating agents, disintegrating agents, fillers and diluents, flavoring agents, colorants, glidants, lubricants, preservatives, sorbents, sweeteners and combinations thereof.

In still another embodiment of the present invention, the composition is formulated into dosage forms selected from a group comprising liquid, troches, lozenges, powder, granule, capsule; tablet, patch, gel, emulsion, cream, lotion, dentrifice, drop, suspension, syrups, elixirs, phyotceuticals and neutraceuticals.

The present invention also relates to a process for preparing synergistic composition comprising boswellic acid fraction at a concentration of about 60% and polysaccharide fraction at a concentration of about 40% optionally along with pharmaceutically acceptable excipients, said process comprising steps of:
obtaining boswellic acid fraction and polysaccharide fraction from Boswellia species;
combining the boswellic acid fraction at a concentration of about 60% to and the polysaccharide fraction at a concentration of about 40% optionally along with pharmaceutically acceptable excipients to obtain the composition.

In still another embodiment of the present invention, the boswellic acid fraction comprise β-boswellic acid, acetyl-β-boswellic acid, 11-keto-β-boswellic acid and acetyl-11-keto-β-boswellic acid.

In still another embodiment of the present invention, the polysaccharide fraction comprise galactose, arabinose, D-glucuronic acid and 4-O-methyl-glucuronoarabino-galactan.

The present invention relates to a method for down-regulating/inhibiting pro-inflammatory markers, said method comprising step of administering a composition comprising boswellic acid fraction at a concentration of about 60% and polysaccharide fraction at a concentration of about 40% optionally along with pharmaceutically acceptable excipients to a subject in need thereof.

In still another embodiment of the present invention, the boswellic acid fraction and the polysaccharide fraction are obtained from Boswellia species.

In still another embodiment of the present invention, the subject is animal including human beings.

In still another embodiment of the present invention, the pro-inflammatory markers are selected from a group comprising TNF-α, IL-β, IFN-γ, nitric oxide and LTB4.

The present invention relates to a method for down-regulating/inhibiting PGE2, said method comprising step of administering composition containing polysaccharide fraction alone or in combination with boswellic acid fraction optionally along with pharmaceutically acceptable excipients to a subject in need thereof.

In still another embodiment of the present invention, the boswellic acid fraction and the polysaccharide fraction are obtained from Boswellia species.

In still another embodiment of the present invention, the subject is animal including human beings.

The present invention also relates to a dietary supplement containing composition mentioned above either singly or in combination.

The current invention represents an improvement over existing conventional Boswellia extracts providing manufacturers with a more water soluble version with enhanced joint health support potential. The composition offers a unique release profile for the active ingredients. In addition to boswellic acids, (the active principles for which Boswellia extracts are conventionally standardized), the polysaccharide fraction from the gum resin of Boswellia serrata also has bioactivity and is water soluble. The polysaccharide fraction enhances the healthful role of boswellic acids in the extract, as revealed in vitro and in vivo studies, at levels beyond a merely additive expectation.

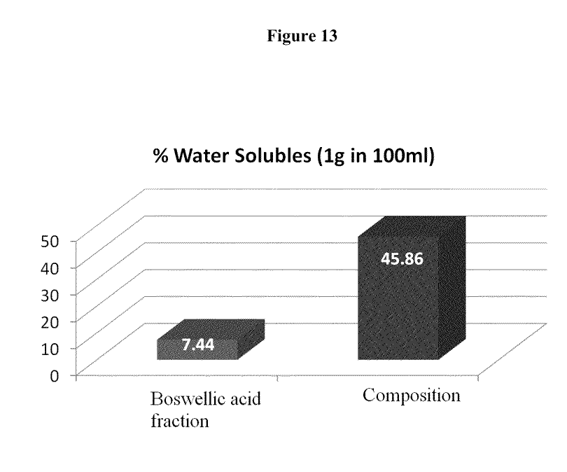

The composition comprising boswellic acid fraction and polysaccharide fraction obtained from Boswellia species shows down-regulation of pro-inflammatory markers. The composition shows enhanced activity in down-regulating pro-inflammatory cytokines or mediators as compared to individual components of composition and hence is synergistic in nature. The polysaccharide fraction of Boswellia species is a water soluble active and thereby increases the solubility of boswellic acids (FIG. 13), reduces the toxicity of boswellic acids at higher concentration and allows sustained anti-inflammatory action.

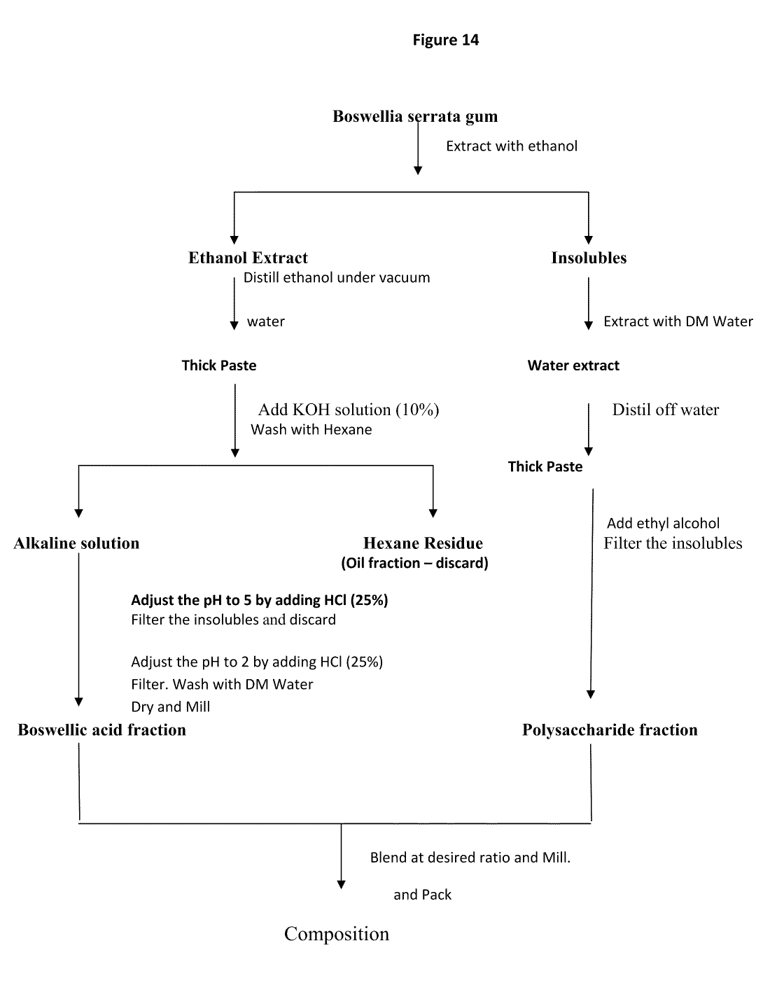

Boswellia gum is extracted with ethanol and the ethanol extract is treated with acid-base followed by water wash to give boswellic acid fraction. Hexane residue (oil fraction) obtained in the process is discarded. The marc remaining after extraction of Boswellia gum with ethanol is extracted with distilled water and precipitated with alcohol to obtain the polysaccharide fraction. The boswellic acid fraction and the polysaccharide fraction are combined at a concentration of about 60% and 40% respectively to arrive at the composition of present invention (FIG. 14).

The boswellic acid fraction comprises β-boswellic acid, acetyl-β-boswellic acid, 11-keto-β-boswellic acid and acetyl-11-keto-β-boswellic acid. The polysaccharide fraction comprises galactose, arabinose, D-glucuronic acid and 4-O-methyl-glucuronoarabino-galactan.

The composition may optionally contain pharmaceutically acceptable excipients selected from a group comprising antiadherents, binding agents, coating agents, disintegrating agents, fillers and diluents, flavoring agents, colorants, glidants, lubricants, preservatives, sorbents, sweeteners and combinations thereof.

The composition of the present invention is formulated into dosage forms selected from a group comprising liquid, troches, lozenges, powder, granule, capsule, tablet, patch, gel, emulsion, cream, lotion, dentrifice, drop, suspension, syrups, elixirs, phyotceuticals and neutraceuticals.

The composition was tested for its potential to inhibit/down-regulate/decrease the levels of pro-inflammatory markers such as TNF-α, IL-β, nitric oxide, IFN-γ, PGE2 and LTB4.

TNF-α (tumor necrosis factor-alpha) is a cytokine involved in systemic inflammation and stimulates the acute phase reaction. Regulation of TNF production has been implicated in a variety of human diseases, as well as cancer. It plays a very important role in the pathogenesis of septic shock induced by LPS.

IL-1β is one of the most potent pro-inflammatory cytokines involved both in physiological immune responses and in the development of various immunopathological disorders. Checking IL-1β levels is useful in monitoring and diagnosis of various diseases including inflammatory, immunological and bone diseases.

Nitric oxide: Appropriate levels of Nitric Oxide production are important in protecting an organ such as the liver from ischemic damage. However, sustained levels of NO production result in direct tissue toxicity and contribute to the vascular collapse associated with septic shock. Chronic expression of NO is associated with various carcinomas and inflammatory conditions including juvenile diabetes, multiple sclerosis, arthritis and ulcerative colitis.

By down-regulating/decreasing the cytokines or other mediators, the composition finds potential use in the management of various diseases/disorders showing elevated levels of these such as Arthritis, Ulcerative Colitis, Inflammatory Bowel syndrome (IBD), Asthma (Respiratory disorders) etc.

The invention is further elaborated with the help of following examples. However, these examples should not be construed to limit the scope of invention.

### Example 1

### Biological Activity Evaluation

### Acute Safety Study:

The acute oral toxicity studies were carried out following OECD guidelines No. 423 [Organization for Economic Cooperation and Development. OECD guidelines for testing of chemicals. Guideline 423, acute oral toxicity—acute toxic class method, adopted, Mar. 22, 1996] in mice. The animals were observed individually periodically during the first 24 h, with special attention given during the first 4 h, and daily thereafter, for a total of 14 days, simultaneously. A single dose of 2000 mg/kg p.o. of the composition administered orally to each group of female mice did not show any change in gross general behaviour of these test animals. A single dose of 5000 mg/kg p.o. was also evaluated. No mortality or any change in normal behaviour when compared to the vehicle controlled group of experimental animals was observed at this high oral dose.

### In-Vitro Studies

### Example 1

### Intracellular In Vitro TNF-α Estimation in Murine Neutrophils

The test materials were subjected to in vitro study, whereby, flowcytometric studies were carried out to determine the effect of multiple doses of boswellic acids fraction, polysaccharide fraction and the composition of invention on intracellular TNF-α cytokine expression in murine neutrophils separated from the whole blood by histopaque gradient.

Cells were stimulated with LPS and incubated with test materials at graded concentrations (μg/ml) for 3 hours, in a CO₂ incubator. The permeabilising solution was added to the cells and then these were incubated for 10 min. The cells were then labeled with conjugated anti-mouse TNF-α monoclonal antibody and further incubation of 30 min. duration was carried out in dark. After washing with Phosphate buffered saline, the samples were acquired directly on BD-Cantoll Flowcytometer (Beckton-Dickinson Biosciences, CA, USA). A fluorescence trigger was set on the FL1 parameter of the gated neutrophils populations (10,000 events) and fluorescence compensation, data analysis and data presentation was performed using Cell Quest Pro software. [Clara, B., R. C. Arancha, G. M. Andre's, P. Atanasio, A. Julia, and O. Alberto. 2003. A new method for detecting TNF-α-secreting cells using direct immunofluorescence surface membrane stainings. J. Immuno. Methods 264:77-87.][Khurshid A. Bhat, Bhahwal A. Shah, Kuldeep K. Gupta, Anjali Pandey, Sarang Bani, Subhash C. Taneja. Semi-synthetic analogs of pinitol as potential inhibitors of TNF-a cytokine expression in human neutrophils. Bioorganic & Medicinal Chemistry Letters 19 2009, 1939-1943]. From the results given in Tables 1, 2 & 3 (flowcytometric studies), it is clear that the composition displayed maximum inhibitory effect on TNF-α cytokine secretion in murine isolated neutrophils in response to LPS stimulant when compared to that of boswellic acid fraction and polysaccharide fraction alone. Neutrophils treated in vitro with 25, 50, 100, 200, 400 and 800 μg/ml concentrations of boswellic acids fraction, polysaccharide fraction and the composition showed 30.52%, 29.31% and 59.83% TNF-α suppression at the dose level of 200 μg/ml respectively. It is clearly evident from the data that the composition at the same dose level showed enhanced activity in suppressing TNF-α as compared to individual fractions.

TABLE 1
Effect of multiple dose of boswellic acid fraction
obtained from Boswellia species on expression
of intracellular TNF-α in murine neutrophils

| | | | | % TNF-alpha |
|---|---|---|---|---|
| | | Concen- | | expression |
| S. | | tration | | against LPS |
| No. | Samples | (μg/ml) | Mean ± S.E | control |
| 1 | LPS Control | — | 2.49 ± 0.02 | — |
| 2 | Boswellic acid | 25 | 2.14 ± 0.03 | 14.05↓ |
| | fraction | | | |
| 3 | Boswellic acid | 50 | 2.09 ± 0.04 | 16.06↓ |
| | fraction | | | |
| 4 | Boswellic acid | 100 | 1.81 ± 0.03* | 27.30↓ |
| | fraction | | | |
| 5 | Boswellic acid | 200 | 1.73 ± 0.01* | 30.52↓ |
| | fraction | | | |
| 6 | Boswellic acid | 400 | 1.46 ± 0.03** | 41.36↓ |
| | fraction | | | |
| 7 | Boswellic acid | 800 | + | + |
| | fraction | | | |
| 8 | Rolipram | 100 | 0.71 ± 0.04** | 71.48↓ |
| | (standard) | | | |

No. of Observations-3;
↓- Decrease in intracellular TNF alpha expression in murine neutrophil;
p value
*<0.01;
**<0.001;
+: cell death

TABLE 2
Effect of multiple dose of polysaccharide fraction
obtained from Boswellia species on expression
of intracellular TNF-α in murine neutrophils

| | | | | % TNF-alpha |
|---|---|---|---|---|
| | | Concen- | | expression |
| S. | | tration | | against LPS |
| No. | Samples | (μg/ml) | Mean ± S.E | control |
| 1 | LPS Control | — | 2.49 ± 0.02 | — |
| 2 | Polysaccharide | 25 | 2.19 ± 0.05 | 12.04↓ |
| | fraction | | | |
| 3 | Polysaccharide | 50 | 2.05 ± 0.02 | 17.67↓ |
| | fraction | | | |
| 4 | Polysaccharide | 100 | 1.88 ± 0.01* | 24.49↓ |
| | fraction | | | |
| 5 | Polysaccharide | 200 | 1.76 ± 0.02* | 29.31↓ |
| | fraction | | | |
| 6 | Polysaccharide | 400 | 1.87 ± 0.05* | 24.89↓ |
| | fraction | | | |
| 7 | Polysaccharide | 800 | 1.90 ± 0.02 | 23.69↓ |
| | fraction | | | |
| 8 | Rolipram | 100 | 0.71 ± 0.04** | 71.48↓ |
| | (standard) | | | |

No. of Observations-3;
↓- Decrease in intracellular TNF alpha expression in murine neutrophils
p value
*<0.01;
**<0.001;

TABLE 3
Effect of multiple dose of the composition of present invention
on expression of intracellular TNF-α in murine neutrophils

| | | | | % TNF-alpha |
|---|---|---|---|---|
| | | Concen- | | expression |
| S. | | tration | | against LPS |
| No. | Samples | (μg/ml) | Mean ± S.E | control |
| 1 | LPS Control | — | 2.49 ± 0.02 | — |
| 2 | Composition | 25 | 1.88 ± 0.07 | 24.49↓ |
| 3 | Composition | 50 | 1.85 ± 0.02* | 25.70↓ |
| 4 | Composition | 100 | 1.43 ± 0.03** | 42.57↓ |
| 5 | Composition | 200 | 1.00 ± 0.01** | 59.83↓ |
| 6 | Composition | 400 | 1.23 ± 0.05** | 50.60↓ |
| 7 | Composition | 800 | 1.51 ± 0.01** | 39.35↓ |
| 8 | Rolipram | 100 | 0.71 ± 0.04** | 71.48↓ |
| | (standard) | | | |

No. of Observations-3;
↓- Decrease in intracellular TNF alpha expression in murine neutrophils
p value
*<0.01;
**<0.001;

### In-Vivo Studies

### Example 2

### Extracellular In Vivo TNF-α, IL-1 Beta and Nitric Oxide (NO) Estimation in Serum from the Treated Mice

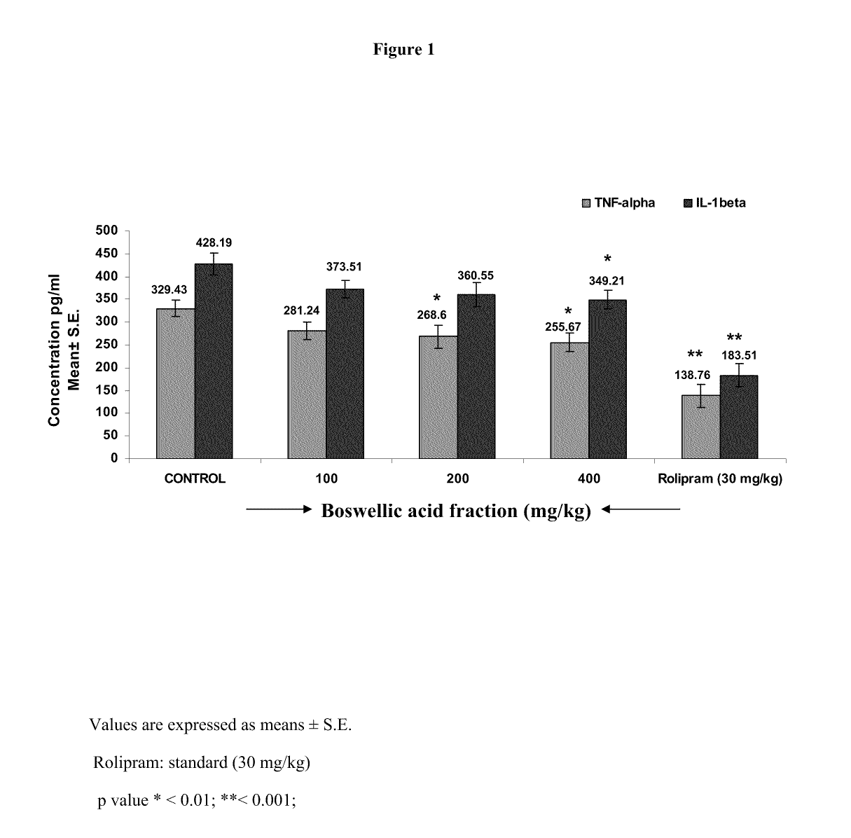

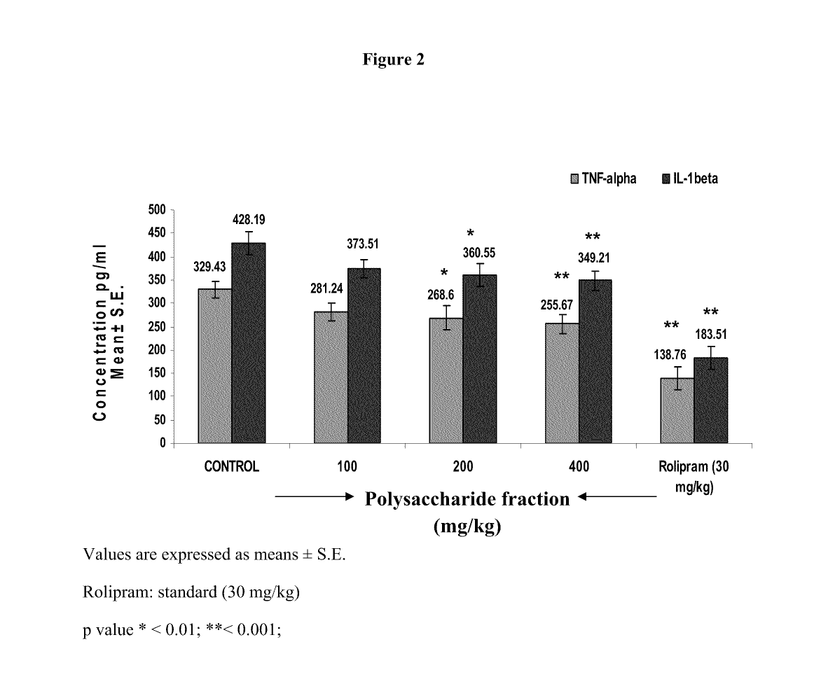

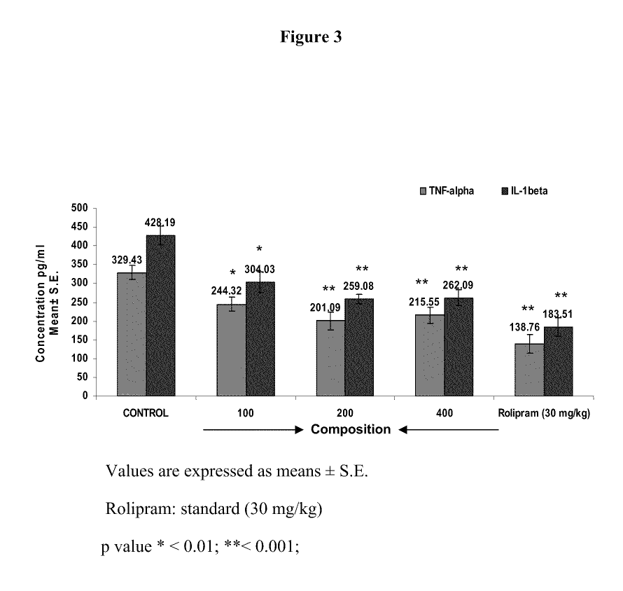

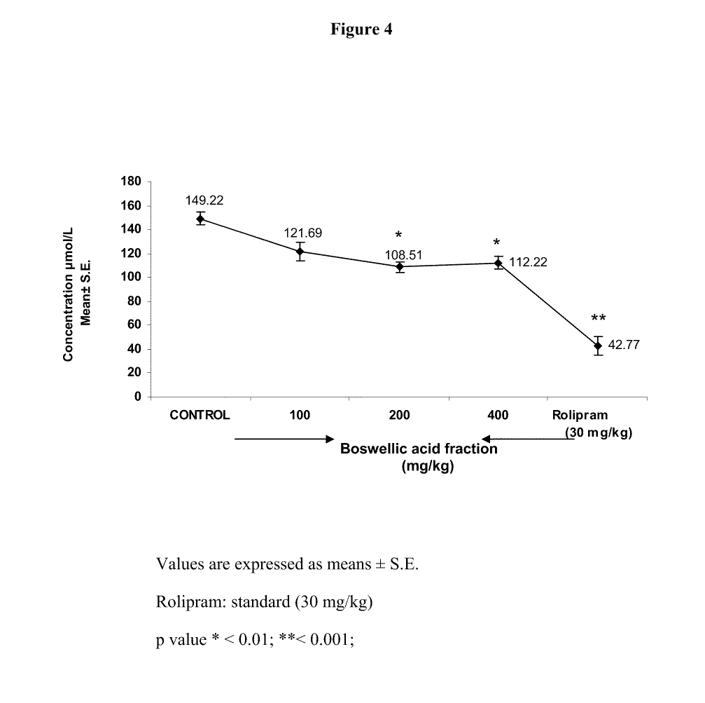

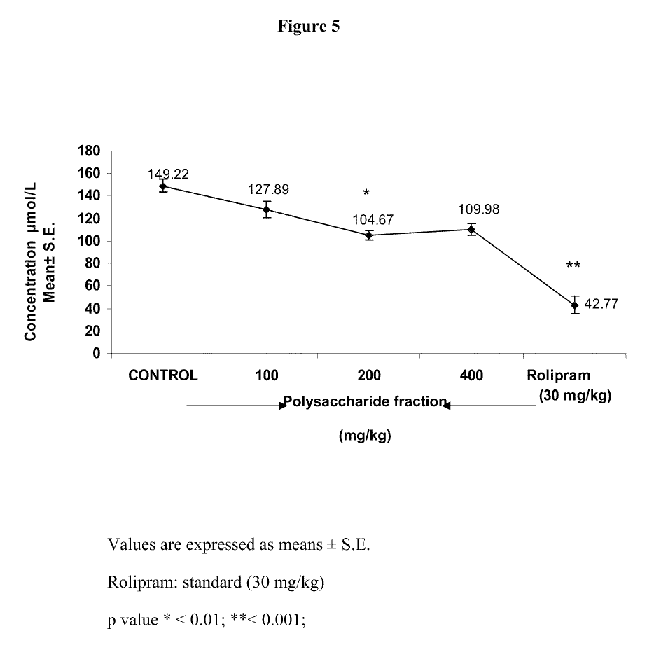

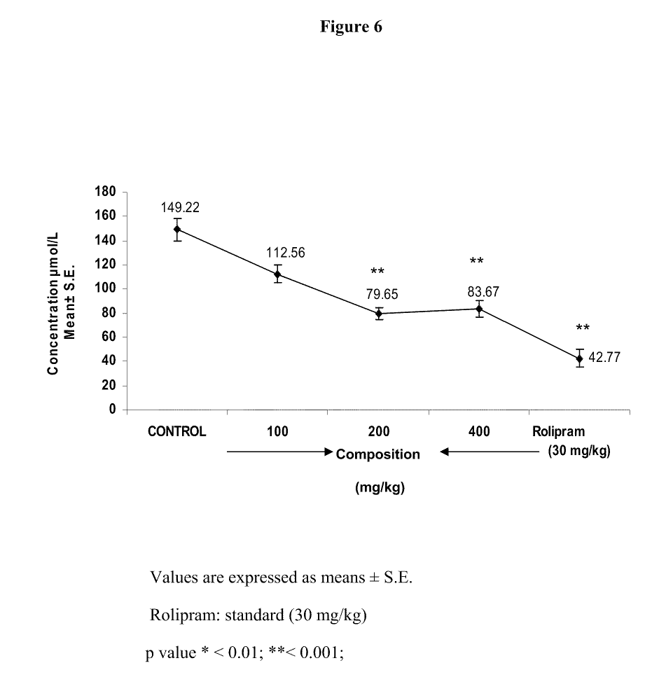

BALB/c male mice aged 6-8 weeks were maintained at 22±2° C. under 12/12 h light dark cycle. Mice received oral treatment of 100, 200, 400 mg/kg of different test materials (w/v) i.e. Boswellic acid fraction, polysaccharide fraction and the composition of present invention for 6 days, followed by intravenous injection of 1 mg/kg of LPS according to the method described by Brieva et al., 2001. [Brieva A, Guerrero A, Alonso-Lebrero J L and Pivel J P. 2001. Immunoferon, a glycoconjugate of natural origin, inhibits LPS-induced TNF-a production and inflammatory responses. International Immunopharmacology 1.1979-1987]. Six mice were employed in each group and experiments were performed in triplicates. TNF-α, IL-1 beta and Nitric oxide production was evaluated by a commercial ELISA kits (R&D Systems) in serum from every experimental group treated mice, 90 min after LPS injection. Rolipram at 30 mg/kg was used as standard drug. Serum collection and measurements revealed a significant reduction in the levels of serum TNF-α, IL-1 beta and NO which suggests a broad species-independent in vivo efficacy for boswellic acid fraction, polysaccharide fraction and the composition in the control of the inflammatory response. Together, these data suggest a regulatory role of the composition in response to increased LPS concentration in blood, not only in the TNF-α production level, but was further confirmed by reduced levels of IL-1 beta, another pro inflammatory cytokine, and NO in LPS-challenged mice, more significant than observed for boswellic acid fraction and polysaccharide fraction alone (FIGS. 1-6). Rolipram at 30 mg/kg dose level was used as a standard drug to observe the authenticity and reproducibility of the experimental design.

In order to show that the composition works in a diseased condition, the following study was conducted. The diseased condition selected for the study is arthritis.

### Example 3

### Adjuvant-Induced Developing Inflammatory Arthritis

Wistar rats, 12-14 weeks old, 140-160 g body weight in groups of 6 were employed in the study. All the animals were maintained in plastic cages at 22±2° C. with 12 h light/dark cycle and free access to pellet food and water. Test materials were administered orally once a day for the duration of the experiment. In all the experiments, a control group was maintained (vehicle administered) whilst the other group received a standard drug acetylsalicylic acid (ASA) administered once daily for comparison and authenticity/credibility of the test. The entire study was carried out after approval from Institutional Animal Ethics Committee and all the animals used in experimental work received humane care. The mean and standard error (S.E.) of the mean for each group was calculated and the results were expressed as percent inhibition compared to control group. The significance was determined statistically by applying Student's t-test.

Adjuvant arthritis was induced by the sub-plantar injection of 0.05 ml freshly prepared suspension (5.0 mg/ml) of steam killed Mycobacterium tuberculosis in liquid paraffin [Newbould BB. Chemotherapy of arthritis induced in rats by mycobacterial adjuvant. Br J Pharmacol 1963; 21:127-36]. The volume of the injected paws was quantitated before and on day 14th after the adjuvant injection and was measured by a volume differential meter model LE 7500N, Panlab, Spain.

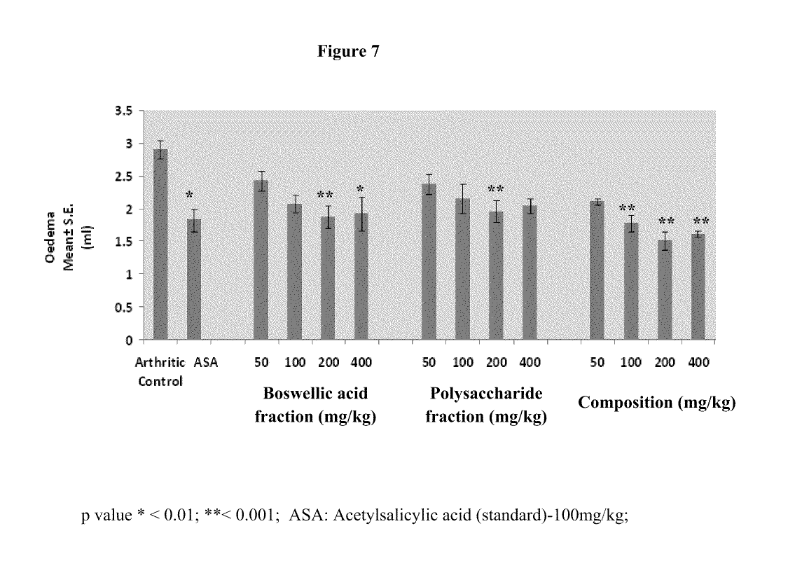

Boswellic acid fraction, polysaccharide fraction and the composition showed dose dependent inhibition of Oedema at dose level of 200 mg/kg orally (FIG. 7 and Table 4). The composition showed highly significant activity of 48% inhibition of Oedema when compared to control in Mycobacterium tuberculli induced inflammatory arthritis in rats.

TABLE 4
Comparative anti-arthritic activity (prophylactic) of boswellic
acid fraction, polysaccharide fraction and the composition of
present invention in Mycobacterium tuberculli induced
inflammatory arthritis in rats (injected paw)

| | | Oedema | Percentage |
|---|---|---|---|
| | Dose | Mean ± S.E. | Activity Against |
| Drug/Treatment | (mg/kg) | (mm) | Arthritic Control |
| Arthritic Control | | 2.9 ± 0.14 | |
| ASA | | 1.82 ± 0.17** | 37% ↓ |
| Boswellic acid | 50 | 2.42 ± 0.15 | 17% ↓ |
| fraction | 100 | 2.07 ± 0.13 | 29% ↓ |
| | 200 | 1.87 ± 0.17** | 36% ↓ |
| | 400 | 1.92 ± 0.26* | 34% ↓ |
| Polysaccharide | 50 | 2.37 ± 0.15 | 18% ↓ |
| fraction | 100 | 2.15 ± 0.22 | 26% ↓ |
| | 200 | 1.95 ± 0.17** | 33% ↓ |
| | 400 | 2.04 ± 0.11 | 30% ↓ |
| Composition | 50 | 2.1 ± 0.05 | 28% ↓ |
| | 100 | 1.77 ± 0.12** | 39% ↓ |
| | 200 | 1.5 ± 0.14** | 48% ↓ |
| | 400 | 1.61 ± 0.05** | 44% ↓ |

ASA: Acetylsalicylic acid (standard)-100 mg/kg;
↓: percent inhibition
p value
*<0.01;
**<0.001.

### Example 4

### Homogenization of Developing Inflammatory Arthritic Paw Tissue on Day 14

Before homogenization for each assay, frozen paw containing bony tissue was weighed and broken into pieces on dry ice. Paw tissues were added to 4 ml/g tissue of extraction buffer containing 1 mM phenylmethylsulfonyl fluoride, 1 mg/ml aprotinin, and 0.05% Tween 20 in phosphate buffered saline. Tissues were homogenized on ice with a polytron and homogenate was centrifuged at 5000 g for 15 min. Supernatants were stored at −80° C. until analysis. [Anjali Pandey, Sarang Bani, Prabhu Dutt, Krishna Avtar Suri. Modulation of Th1/Th2 cytokines and inflammatory mediators by hydroxychavicol in adjuvant induced arthritic tissues; Cytokine 49 (2010) 114-121].

Quantification of TNF-α, PGE₂ and LTB₄ in Supernatant from Tissue Homogenate:

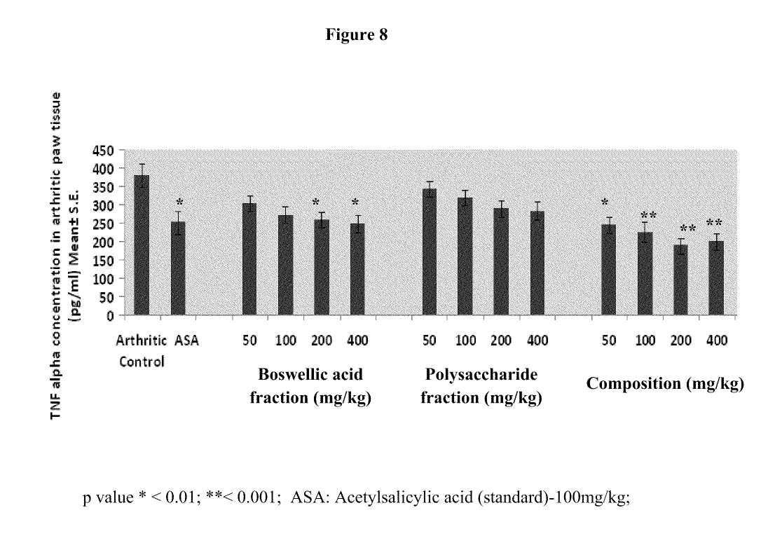

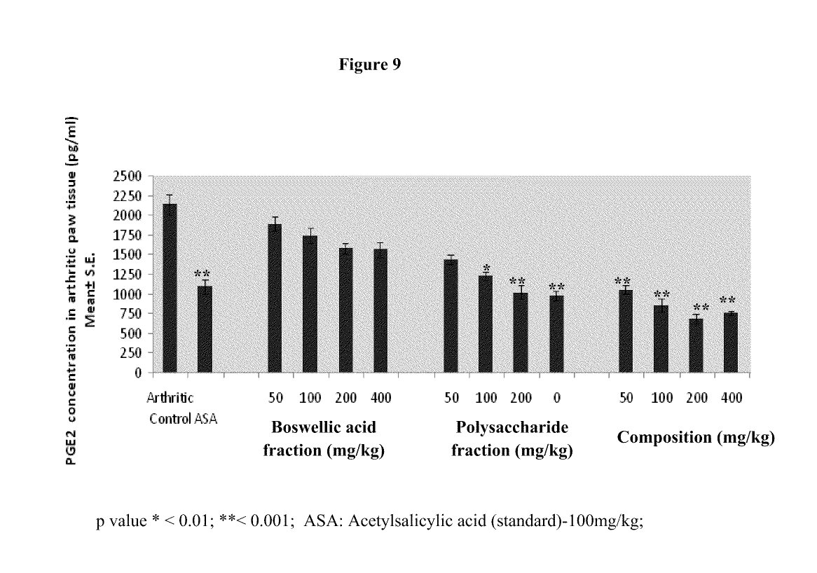

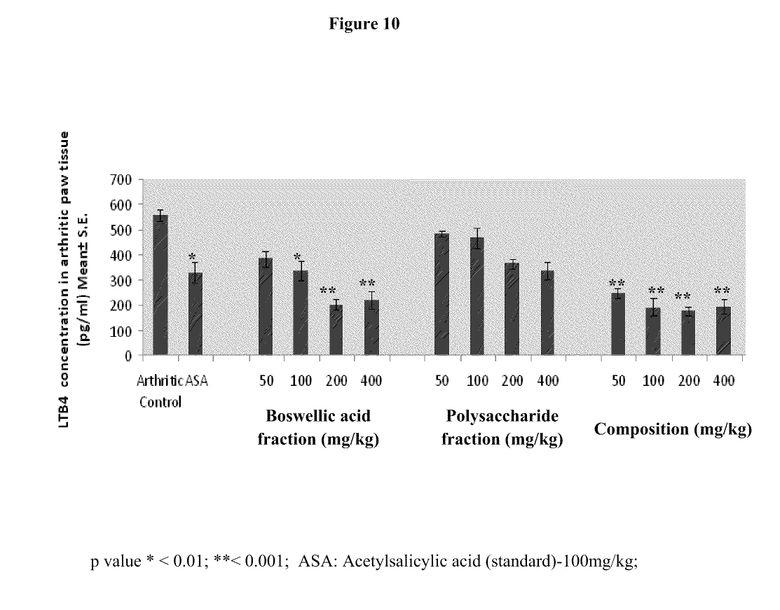

Samples on day 14 from different groups of animals were prepared for the analysis of cytokine mediators as described above. TNF-α, PGE₂ and LTB₄ were estimated using commercially available kits based on sandwich and competitive ELISA technique (R&D Systems, MN, USA) according to the manufacturers' instructions. All cytokine concentrations were carried out by means of colorimetric measurement at 450 nm on an ELISA plate reader (Multiskan, Thermo Electron Corporation, MA, USA) by interpolation from a standard curve. [Anjali Pandey, Sarang Bani, Prabhu Dutt, Krishna Avtar Suri. Modulation of Th1/Th2 cytokines and inflammatory mediators by hydroxychavicol in adjuvant induced arthritic tissues; Cytokine 49 (2010) 114-121][Magari K, Miyata S, Ohkubo Y, Mutoh S. Inflammatory cytokine levels in paw tissues during development of rat collagen-induced arthritis: effect of FK506, an inhibitor of T cell activation. Inflamm Res 2004; 53:469-74]. Boswellic acid fraction showed significant decrease of TNF-α and LTB4 but no effect on PGE2 in arthritis affected animals. Polysaccharide fraction showed moderate decrease of TNF-α and PGE₂ levels with no significant inhibition of LTB₄ whereas the composition significantly decreased the TNF-α, PGE2 and LTB4 levels in a dose dependent manner showing maximum inhibition at an oral dose of 200 mg/kg dose (FIGS. 8, 9 and 10).

The finding that the polysaccharide fraction showed inhibitory effect on PGE2 is a surprising finding of the present invention and hence is novel. It is evident from the present study that the polysaccharide fraction alone or in combination with boswellic acids is useful in inhibiting PGE2 at moderate levels unlike other drugs known in the art having side-effects due to high levels of PGE2 inhibition.

### Example 5

### Intracellular IFN-γ Detection by Flowcytometry in Splenocytes

The etiologic cause of arthritis has not been clearly delineated but cumulative evidence suggests that T cell-mediated autoimmune responses play a critical role in its pathogenesis [Panayi G S. T cell-dependent pathways in rheumatoid arthritis. Cur Opin Rheumatol 1977; 9:236-40]. To increase the specificity of therapies for arthritis, emphasis has shifted to targeting cytokines. IFN-producing Th1 cells appear to be pivotal in the development of arthritis in both humans and animal models [Garra O. Cytokines induce the development of functionally heterogeneous T helper cell subsets. Immunity 1998; 8:275-83]. Thus, recent therapeutic strategies have focused on modulating the response of Th1 cells. The pharmacodynamic studies indicate Th1/Th2 modulation as possible mechanism of action for cytokine therapy in many diseased conditions [Lissoni P, Malugani F, Malysheva O. Neuroimmunotherapy of untreatable metastatic solid tumors with subcutaneous low-dose, interleukin-2, melatonin and naltrexone, modulation of interleukin-2-induced antitumor immunity by blocking the opoid system. Neuroendocrinol Lett 2002; 23: 341-4.] [Tabata N, Tagami H, Terui T. Dehydroepiandrosterone may be one of the regulators of cytokine production in atopic dermatitis. Arch Dermatol Res 1997; 289: 410-4.] Cell mediated immune responses play an important role during the development of arthritis [Waksman B H, Pearson C M, Sharp J T. Studies of arthritis and another lesions induced in rats by injection of mycobacterial adjuvant-II: evidence that the disease is a disseminated immunologic response to exogenous antigen. J Immunol 1960; 85:403-17] and inhibition of this response particularly IFN-γ produced by CD4+ T cells show a strong correlation of composition having anti-arthritic activity. The composition produced a dose related inhibition of IFN-γ produced by CD4+ T cells.

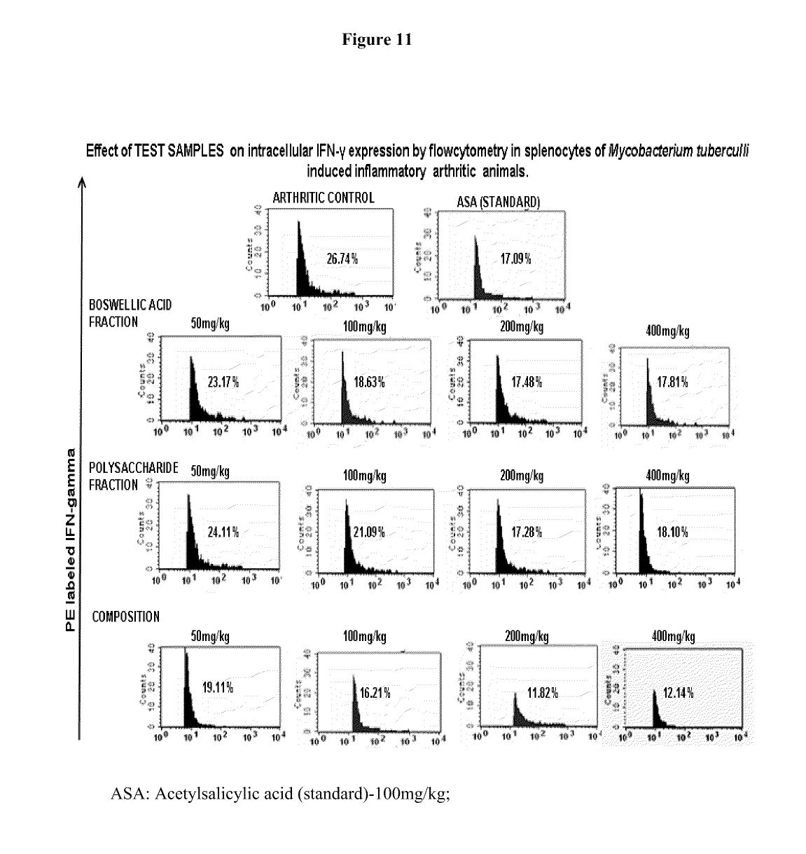

Spleen was collected from animals of all the test groups under aseptic conditions, in Hank's balanced salt solution (HBSS, Sigma to obtain a homogeneous cell suspension, and the erythrocytes were lysed with FACS Lysing solution. After centrifugation (380 g at 4° C. for 10 min), the pelleted cells were washed three times in PBS and resuspended in complete medium [RPMI 1640 supplemented with 12 mM Hepes (pH 7.1), 0.05 mM 2-mercaptoethanol, 100 IU/ml penicillin, 100 Ig/ml streptomycin, and 10% FCS]. Cell numbers were counted with a hemocytometer by trypan blue dye exclusion technique. Cell viability exceeded 95%. Briefly, splenocytes were seeded into 96-well flat-bottom microtiter plate (Nunc) at 2×10⁶ cells/ml. After 3 days, splenic lymphocytes were stained with 5 μl PE conjugated anti-mouse IFN-γ antibody and incubated for 30 min at 4° C. in the presence of 1 μl FACS permeabilizing solution. Analysis was carried out on the flowcytometer (BD, LSR) using the Cell Quest Pro software. [Anjali Pandey, Sarang Bani, Prabhu Dutt, Krishna Avtar Suri. Modulation of Th1/Th2 cytokines and inflammatory mediators by hydroxychavicol in adjuvant induced arthritic tissues; Cytokine 49 (2010) 114-121]. We cultured the splenocytes with fluorochromes to assess intracellular cytokine contents. Predictably we noted a higher percentage expression of 26.74% of IFN gamma in arthritic control group. To clarify the characteristics of IFN gamma related cytokine-producing lymphocyte subsets, we examined IFN gamma producing cells among CD4+ T cells. We noted a rather lower level of intracellular IFN gamma from boswellic acids fraction, Polysaccharide fraction and the composition treated spleenocytes at graded doses. Maximum suppression was observed in the composition treated group at 200 mg/kg p.o. dose (FIG. 11).

### Example 6

### Adjuvant-Induced Established Inflammatory Arthritis

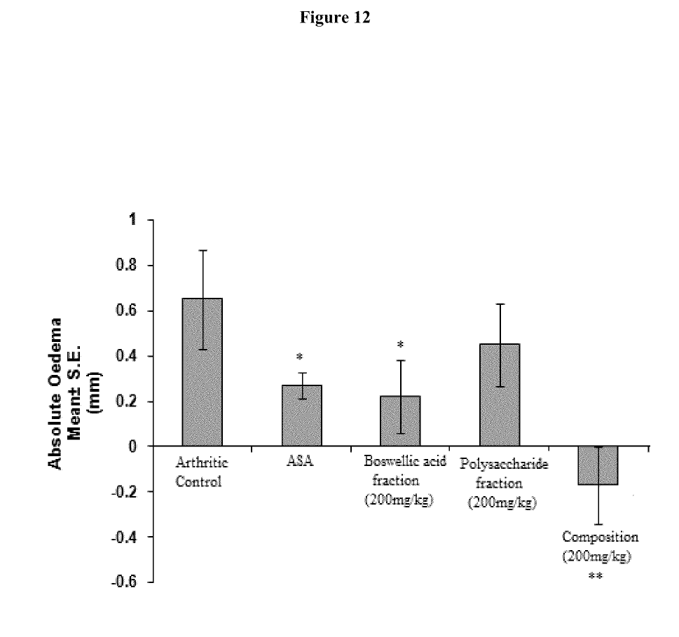

Arthritis is induced by injecting dead Mycobacterium tuberculosis in oil in sub-planter region of left hind paw as mentioned above. Disease develops during first 14 days. No drug is given during this period. From Day 15 the drug is administered orally to the animals up to day 28. This is a test that shows therapeutic potential of the test material in established arthritis [Newbould, B. B., 1969. The pharmacology of fenclozic acid 2-(-4-chlorophenyl)theazol-4-ylactic acid: I.C.I. 54,450; Myalex: a new compound with anti-inflammatory, analgesic and anti-pyretic activity, British journal of Pharmacology. 35, 189-197]. The absolute oedema inhibition was observed in animals treated with boswellic acid fraction, polysaccharide fraction and the composition of present invention. However, the composition showed the most significant effect (inhibition of absolute oedema) in the established arthritic rats (FIG. 12).

Thus, the overall results indicate that the composition of present invention shows significant inhibition of the targets more than that of boswellic acid fraction and polysaccharide fraction alone. The maximum effect in vitro was at 100 and 200 μg/ml and the maximum effect in vivo was at a dose of 100 and 200 mg/kg animal body weight/p.o./day treatment in experimental animals.

The composition is suggested at a dosage of up to 500 mg, 2 to 3 times a day to an individual in need thereof.

1) A synergistic composition comprising boswellic acid fraction at a concentration of about 60% and polysaccharide fraction at a concentration of about 40%. optionally along with pharmaceutically acceptable excipients.

2) The composition as claimed in claim 1, wherein the boswellic acid fraction and the polysaccharide fraction are obtained from Boswellia species.

3) The composition as claimed in claim 1, wherein the boswellic acid fraction comprise β-boswellic acid, acetyl-β-boswellic acid, 11-keto-β-boswellic acid and acetyl-11-keto-β-boswellic acid.

4) The composition as claimed in claim 1, wherein the polysaccharide fraction comprise galactose, arabinose, D-glucuronic acid and 4-O-methyl-glucuronoarabino-galactan.

5) The composition as claimed in claim 1, wherein the pharmaceutically acceptable excipients are selected from a group comprising antiadherents, binding agents, coating agents, disintegrating agents, fillers and diluents, flavoring agents, colorants, glidants, lubricants, preservatives, sorbents, sweeteners and combinations thereof.

6) The composition as claimed in claim 1, wherein the composition is formulated into dosage forms selected from a group comprising liquid, troches, lozenges, powder, granule, capsule, tablet, patch, gel, emulsion, cream, lotion, dentrifice, drop, suspension, syrups, elixirs, phyotceuticals and neutraceuticals.

7) A process for preparing synergistic composition comprising boswellic acid fraction at a concentration of about 60% and polysaccharide fraction at a concentration of about 40% optionally along with pharmaceutically acceptable excipients, said process comprising steps of:
obtaining boswellic acid fraction and polysaccharide fraction from Boswellia species;
combining the boswellic acid fraction at a concentration of about 60% and the polysaccharide fraction at a concentration of about 40% optionally along with pharmaceutically acceptable excipients to obtain the composition.

8) The process as claimed in claim 6, wherein the boswellic acid fraction comprise β-boswellic acid, acetyl-β-boswellic acid, 11-keto-β-boswellic acid and acetyl-11-keto-β-boswellic acid.

9) The process as claimed in claim 6, wherein the polysaccharide fraction comprise galactose, arabinose, D-glucuronic acid and 4-O-methyl-glucuronoarabino-galactan.

10) A method for down-regulating/inhibiting pro-inflammatory markers, said method comprising step of administering a composition comprising boswellic acid fraction at a concentration of about 60% and polysaccharide fraction at a concentration of about 40% optionally along with pharmaceutically acceptable excipients to a subject in need thereof.

11) The method as claimed in claim 10, wherein the boswellic acid fraction and the polysaccharide fraction are obtained from Boswellia species.

12) The method as claimed in claim 10, wherein the subject is animal including human beings.

13) The method as claimed in claim 10, wherein the pro-inflammatory markers are selected from a group comprising TNF-α, IL-β, IFN-γ, nitric oxide and LTB4.

14) A method for down-regulating/inhibiting PGE2, said method comprising step of administering a composition comprising polysaccharide fraction alone or in combination with boswellic acid fraction optionally along with pharmaceutically acceptable excipients to a subject in need thereof.

15) The method as claimed in claim 14, wherein the polysaccharide fraction and the boswellic acid fraction are obtained from Boswellia species.

16) The method as claimed in claim 14, wherein the subject is animal including human beings.

17) A dietary supplement containing composition of claim 1 and/or claim 14.
